(19) United States
(12) Patent Application Publication
(10) Pub. No.: US 2009/0215178 A1
(43) Pub. Date: Aug. 27, 2009

(54) Methods to enhance the stability and homogeneity of transgene expression in clonal cell lines

(76) Inventor: Zequn Tang, San Ramon, CA (US)

Correspondence Address: Zequn Tang, 3067 Sorrelwood Dr, San Ramon, CA, 94582, US

(21) Appl. No.: 12/035,437

(22) Filed: Feb. 22, 2008

(51) Int. Cl. C12N 15/85

(52) U.S. Cl. 435/455

(57) ABSTRACT

Strategies for increasing the productivity of recombinant protein expression in isogenic cell-lines. Development of a novel selection system for the improved stability and homogeneity of transgene expression in isogenic cells without the dependence on continuous selective drugs.

### TECHNICAL FIELD

The present invention relates generally to recombination DNA technology. In particular, the invention relates to the development of a novel selection system for the improved stability and homogeneity of transgene expression in isogenic cells.

### BACKGROUND OF THE INVENTION

Genetically engineered cell lines play a pivotal role in the production of bioactive molecules, such as antibodies, cytokines, hormones, recombinant coagulation factors, enzymes and other bioactive recombinant proteins, as well as viral like particles, etc.

Currently, the genetically engineered cell lines are usually produced by integrating transgene expression vectors into the cell genome through either chemical transfection or electroporation methods, the cells harboring the transgene expression vectors were selected by various antibiotics marker carried on the expression vector, such as neomycin, hygromycin, zeocin, puromycin, etc. The desired phenotypes were selected from multiple clones, based on the expression level, stability and growth characters, etc. The phenotypic characters of the integrated transgene expression vector usually showed remarkable variations between isogenic cell lines, ranging from homogenous high expression level to mosaic to complete silencing. More importantly, as long as cell clones with homogenous high expression level were kept under selective pressure, their expression level, growth characters could be maintained over a long period. However, these desirable phenotypes were progressively lost upon withdrawal from selective conditions. Most of the lost of the transgene expression events were attributed to epigenetic events, such as DNA methylation, histone protein modification (acetylation, phosphorylation, ubiquitination) etc. Other factors affecting the expression level of transgene might include process variables such as metabolic state of cell, change of chemical components in the culture medium, temperature, and PH, etc.

Several methods have been developed to improve the stability of transgene expression. One of the methods involved in flanking the transgene vector with insulator sequences or anti-repressor sequences, to prevent the spread of heterochromatin, allowing the expression of the transgenes in all cells. Alternatively, homologous recombination-mediated chromosome engineering methods were used to generate stably transfected isogenic cell lines, using this approach, the transgene expression vector fragments can be targeted at predetermined chromosomal loci in the genome for optimal expression and genetic stability. Some chemical substances such as HDAC inhibitors (Sodium Butyrate, trichostatin A, etc.) have also been explored to counteract the silencing effects. However, none of these approaches can ensure either the long-term stability of the expression of the introduced transgenes or the phenotypic homogeneity of the isogenic clones. Other methods include using dihydroforate reductase (DHFR) deficient cell as the cell substrate for genetic engineering transgene expression cell line, the transfected cell pools were then treated with step-wise increasing of methotrexate concentration to facilitate the DHFR-mediated gene amplification. Other methods including metabolic engineering of cell lines with desired growth characters. However, in all the methods, the expression levels of stable integrated transgenes and homogeneity of the cell clones is critically affected by withdrawal of the selective pressure. These results demonstrated the critical importance for new methods development to generate stable transgene cell clones with sustained, uniform gene expression over long generation of passages in the absence of selective marker drugs.

### DESCRIPTION OF THE FIGURES

FIG. 1 Schematic representation of Essential genes knock-down cassette. The promoter, shRNA sequences including the dsDNA and hairpin loop region, the terminator sequences (“TM”) in the cassette are shown.

FIG. 2 Schematic illustration of Supplementary expression cassette. The enhancer, promoter, 5′UTR, essential gene ORF, 3′ UTR and polyadenation signal region are shown.

FIG. 3 schematic representation of inactivation of endogenous gene by essential gene knock-down cassette, and trans complementation of essential gene from supplementary expression cassette. All three expression cassette are in a single DNA molecules.

FIG. 4 Similar to FIG. 3, schematic representation of inactivation of endogenous gene by essential gene knock-down cassette, and trans complementation of essential gene from supplementary expression cassette. The supplementary expression cassette is physically on same DNA molecules as recombinant protein expression cassettes (for bio-production), while the essential gene knock-down cassette is encoded by separate DNA molecules with distinct selection marker.

### DETAILED DESCRIPTION OF INVENTION

The present invention provides the method for generating genetically engineered cell lines with sustained homogeneity and stable transgenes expression in the presence or absence of selective marker drugs. The mechanism of selective pressure was built in the bioactive protein expression vector systems, and the selective pressure will continuously act on the cell lines without the presence of selection drugs.

The invention provides strategies that can be used to inactivate cell endogenous essential genes for cell substrate growth or survive, meanwhile provide the complimentary protein product in trans from a separate transcript unit. It composed mainly of three cassettes: Essential genes knock-down cassettes which inactivate the endogenous essential gene, Supplementary expression cassettes provide the same essential protein product in trans, and Recombinant protein expression cassette express the gene of interest (bioactive protein) product, the recombinant protein expression cassette can be, but not restricted to, physical located on the same DNA molecules as that of supplementary expression cassette, such that selective pressure will be in favor of cells with high expression form the integrated cassettes. This invention provides a novel approach and detailed method to generating stable, homogenous expression clone for recombinant protein expression.

### Essential Gene Knock-Down Cassettes

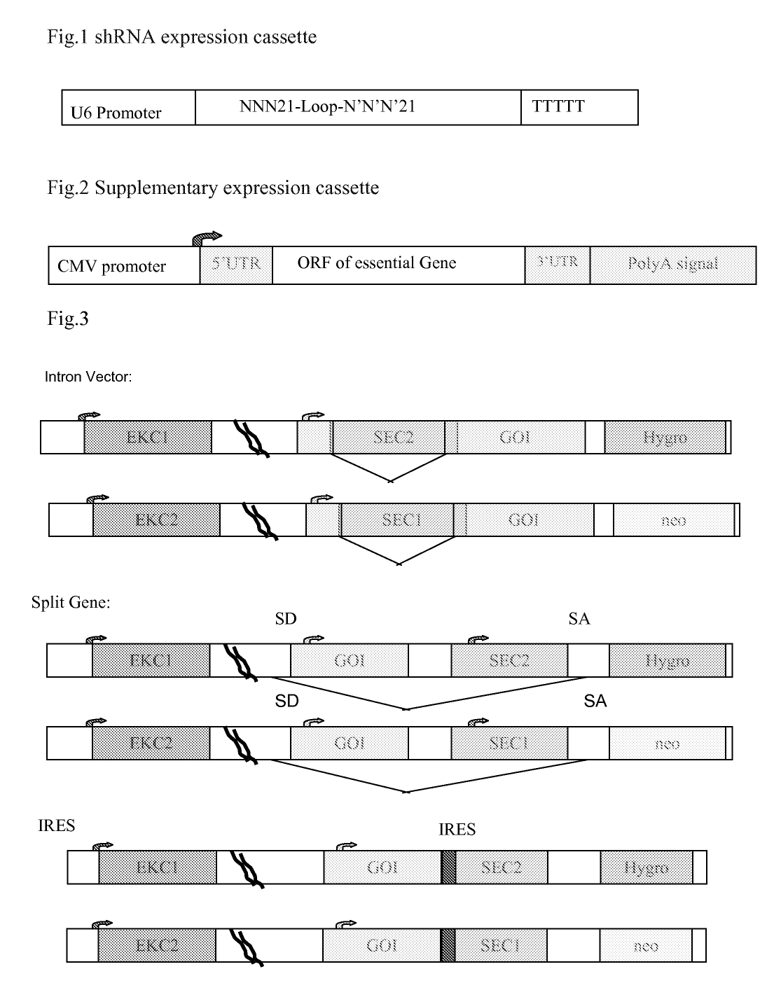

The essential gene knock-down cassettes are comprised of transcription unit/units that capable of inactivating cellular genes essential for cell substrate growth or survive. Such transcription unit usually includes a promoter, RNA transcript coding region containing the sequences capable of knock-down the expression of essential genes for the cell substrate, and might also includes a termination elements or polyadenylation signal element depending on the types of promoters used (FIG. 1).

The examples of essential genes for cell lines survive/proliferation include but not limited to: transforming genes or viruses sequences used to immortalize the cell substrate, such as adenovirus E1a/1b, SV40 large T antigen, Papillomaviruses E6 and E7, Epstein-Barr (EBNA1) virus, human telomerase reverse transcriptase (hTERT). Genes key for cell cycle regulation, such as CCNE1, CDC2, PCNA, MYC, E2F, etc. Genes responsible for dNTP synthesis and chromatin assembly, such as RRM1 RRM2 and SLBP, CHAF1A, etc; Other endogenous cellular essential genes (take human cell as example) include but not limit to: Homo sapiens AD024 (AF225416), Homo sapiens adaptor-related protein complex 1, sigma 1 (NM001283); Homo sapiens ATPase, H+ transporting, lysosomal 34 kDa, V1 subunit D (NM015994); Homo sapiens CASP8 associated protein 2 (NM012115); Homo sapiens cell division cycle 16 homologue (NM003903); Homo sapiens cell division cycle 27 homolog (NM001256); Homo sapiens cDNA FLJ77048 (AK290362); Homo sapiens chemokine-like factor super family 4 isoform 1 (AF521889); Homo sapiens claudin 1 (NM006580); Homo sapiens DEAD (Asp-Glu-Ala-Asp) box polypeptide 5 (NM004396); Homo sapiens DEAD (Asp-Glu-Ala-Asp) box polypeptide 48 (CR456750); Homo sapiens DEAH (Asp-Glu-Ala-His) box polypeptide 8 (NM004941); Homo sapiens mRNA; cDNA DKFZp564MO82 (AL080071); Homo sapiens eukaryotic translation initiation factor 3, subunit 3(CR456906); Homo sapiens EIF3S10 gene for eukaryotic translation initiation factor 3 (AL35598); Homo sapiens cDNA FLJ10290 fis (AK001152); Homo sapiens cDNA FLJ20311 fi (AK000318); Homo sapiens hypothetical LOC653140 (FLJ30851)(NM 001040710); Homo sapiens dynactin 5 (p25) (BC004191); Homo sapiens UDP-N-acetyl-alpha-D-galactosamine: polypeptide N-acetylgalactosaminyltransferase 5 (014568); Homo sapiens heme-regulated eukaryotic initiation factor 2 alpha (AF255050); Homo sapiens inositol 1,4,5-trisphosphate receptor type 1 (L38019); Homo sapiens KIAA0056 (D29954); Homo sapiens kinesin family member 11 (NM004523); Homo sapiens karyopherin (importin) beta 1 (BC036703); Homo sapiens LSM6 homolog (NM007080); Homo sapiens microfibrillar-associated protein 1 (NM005926); Homo sapiens chromosome 1 open reading frame 135 (BC000209); Homo sapiens dynactin 5 (p25) (BC004191); Homo sapiens pleckstrin homology (NM004227); Homo sapiens ribonucleotide reductase M2 polypeptide (NM001034); Homo sapiens squamous cell carcinoma antigen recognized by T cells (NM005146); Homo sapiens small nuclear ribonucleoprotein polypeptide A′ (NM003090); Homo sapiens small nuclear ribonucleoprotein polypeptides B (NM198216); Homo sapiens SNW domain containing 1 (SNW1) (NM012245); Homo sapiens valosin-containing protein (VCP) (NM007126); Homo sapiens zinc finger, DHHC-type containing 5 (NM015457); Other potential target includes nuclear pore protein (NuMA); Nuclear envelop protein (Lamin A/B/C, Nup153);

Cytoskeletal cytoplasmic proteins (actin, vinculin, etc.); Mitotic proteins (Eg5, CENP-E, cdk-1, cytoplasmic dynein); Genes crucial for cell metabolism, such as dihydrofolate reductase, glutamine synthetase, though crucial for animal development, their essential role for survival in cell culture is not certain. It is possible that depends on the cell origin and cell culture environments, different targets might be chosen for maximum efficiency.
Alternatively, combination of nonessential genes, or homologous gene families can be targeted for knocking down to achieve selective advantage for the cell clones harboring supplementary expression cassettes.

The promoters used in the cassette could be of polIII promoters' origin, such U6, H1, 7SK, EBV-encoded small RNAs (EBERs) and adenovirus VAI promoters, etc. The advantages of PolIII promoters include high activity in all cell types, small size of the entire gene knock-down cassette, lack of post-transcriptional modification (such as capping and polyA addition) and confirmed high efficacy in shRNA or dsRNA based gene knock-down. PolII promoter could also be used to driven the expression of knock-down cassette, such as CMV promoter, RSV promoter, retroviral LTR elements, etc. Poll promoter has also been successfully applied to driven the expression of shRNA transcripts.

The RNA transcript region usually contains a shRNA with various hairpin length (such as 19-29 nt) and loop structure. Alternatively, it could also be dsRNA or ribozyme RNA transcripts. The target of the shRNA could be but not limited to either 5′ or 3′ untranslated region of the essential genes as listed, providing that different 5′ and 3′ UTR sequences were used in the supplementary expression cassette. Alternatively targets of shRNAs could also be in the coding region of the endogenous essential genes, if such shRNA target sequences are abolished in the supplementary expression cassette through codon optimization, deletion or insertion in nonessential region of the coding sequences (Examples). Another potential shRNA targets are the promoter regions of endogenous essential genes, since it has been show that that shRNA could potentially induce the heterochromatinization in the target gene promoters.

Multiple essential gene knockdown cassettes could be used for the inactivation procedure, the cassettes could be tandem arranged in the same DNA plasmid (molecule) or on different DNA plasmids with different selective marker. The cassettes could target at various different regions of targeted essential gene, or alternatively it could target at different essential genes.

### Supplementary Expression Cassettes

The supplementary expression cassette usually contains a recombinant DNA molecule comprising of (1) a DNA dependent RNA polymerase promoter, (such as Rous sarcoma virus, MPSV, CMV, EF1-α, VISNA, SV40, etc.) (2) An open reading frame coding for a functional essential gene product as the targeted endogenous essential gene product. (3) The open reading frame of the supplementary essential gene contains different 5′ or 3′ UTR sequences from that of cellular endogenous counter part. Or the coding region has been optimized through mutation, deletion or insertion to abolish the target sequence for the product from essential gene knock-down cassettes (FIG. 2).

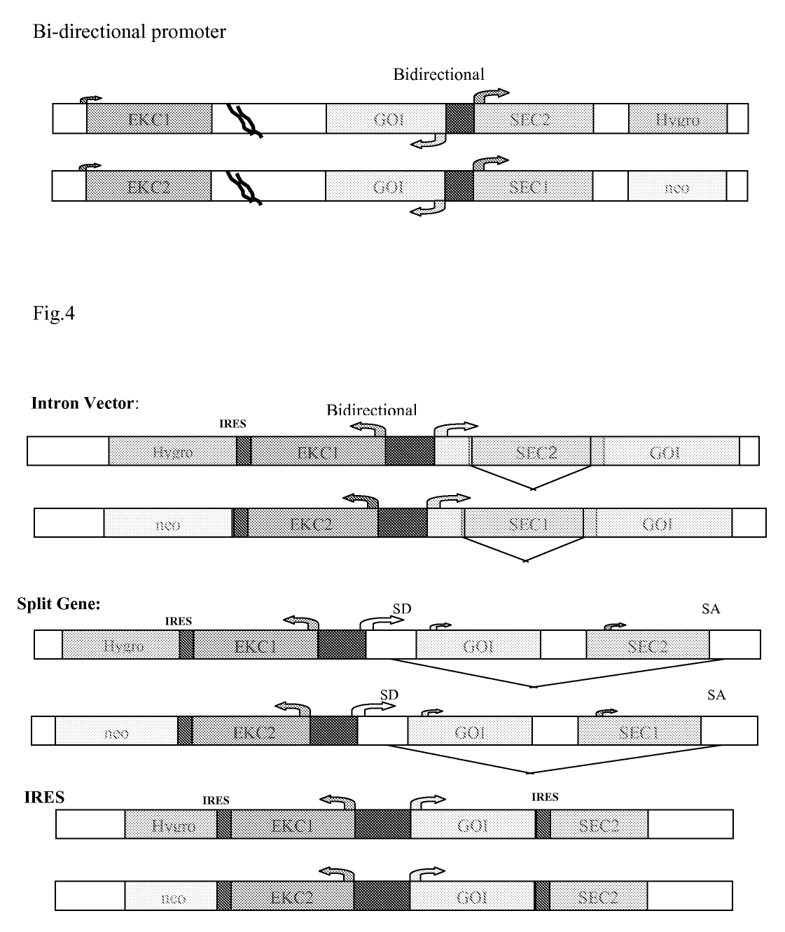

For the maximum efficiency of this system, the supplementary expression cassettes are preferably, but not limited to, physically located at the same DNA molecules (vectors) with that of recombinant protein expression cassette (gene of interest). The two ORFs could be expressed from two separate transcript units, or they could be expressed from a polycistronic transcript, such as through IRES, intron vector, split gene strategy, or expressed from a bidirectional promoter (FIG. 3). The essential gene knock-down cassettes could be physically linked to the other two cassettes or could be expressed from independent DNA molecules (FIG. 4).

### Recombinant Protein Expression Cassette

The current method provides a built in selection mechanism, independent of selection drug, for cell clones harboring recombinant protein expression cassettes. Thus, it could be applied to most of the recombinant expression systems in use today. The recombinant protein expression cassettes consist of, not limited to, polII enhancer/promoter (Rous sarcoma virus, MPSV, CMV, EF1-α, VISNA, SV40, PGK, etc.), an ORF coding for recombinant protein of various applications (antibodies, cytokines, hormones, recombinant coagulation factors, enzymes, viral proteins and other bioactive recombinant proteins, etc.), and the sequences that helps to stabilize the transcripts or enhance the translation of the transcripts.

### Method for Cell Clone Selection

The cassettes embodied by current invention can be introduced into host cells. Host cells can be of any eukaryotic cell suitable for recombinant protein production. These include, but not limit to, insect cells, vertebrate and mammalian cells.

The cassettes embodied by current invention can be introduced into host cells using methods well known in the art, including, but not limited to, microinjection, liposome-mediated transfection, electroporation, and calcium phosphate precipitation. These cassettes can be incorporated into a polynucleotide delivery vehicle, such as plasmid or a viral-based vector.

For cell substrates (such as PerC.6, HEK293, Namalwa cells, etc.) transformed by viral genes such as adenovirus E1a/1b, SV40 large T antigen, Papillomaviruses E6 and E7, Epstein-Barr (EBNA1) virus, etc. The most convenient way of generating stable clone is to use essential gene knock-down cassettes targeting at corresponding viral protein coding sequences. For continuous cell lines derived from stem like cells or obtained by in vitro transformation in cell culture (such as EB-x, MDCK, BHK21, CHO, vero, MRC, etc.), or continuous cell lines derived from tumor cells (such as Hela, Bowes Melanoma, Murine Hybridoma, nonsecreting myeloma cell, etc.), genes crucial for cell survive or cell proliferation as listed above, provide more feasible approach. These targets could be used for all types of cell substrates.

There are several potential ways to select the desired cell clones. In the simplest situation, all three cassettes are located on single DNA molecule (Vector) with proper selection marker, and the complementary gene knocking down cassette and supplementary cassette were located on separate DNA molecules. Two or more DNA molecules needed to be introduced into single cells. DNA was introduced by either electroporation or chemical transfection, the cell clones harboring the vector were selected by proper antibiotics carried on the vector. Cell clones were then selected based on the desired phenotypes.

Alternatively, DNA molecules carrying with supplementary expression cassettes and recombinant protein expression cassettes were introduced first, and selected with proper antibiotics, the clones with desired phenotypes were selected based on the expression level, stability and growth characters, etc. The resulting clones were used to introduce DNA molecules carrying essential gene knock-down cassettes with a different selective marker, and then subjective to further selection.

The efficacy of essential gene knock down cassettes have been pre-examined in various cell lines, based on mRNA, protein level analysis, cell growth characters, etc. For real-time analysis of shRNA efficacy against target sequences, report vectors have been constructed. Basically, a shRNA target sequences was inserted into the 5′UTR of reporter gene (GFP, dsRed, b-Gal, etc.), the level of reporter gene was compared with the similar vector without shRNA target sequences. The reporter constructs were transiently introduced into the selected cell clones and level of expression were examined.

### EXAMPLES

The following examples are provide with the current invention, and should not be interpreted as limiting thereof.

### Example 1

Essential gene knock-down cassette and supplementary expression cassette for AURKB gene. The gene knock-down cassette was targeted at 3′UTR of endogenous AURKB, while supplementary AURKB expression cassette use 3′UTR from SV40 PolyA

Endogenous AURKB 3′UTR: sequences:

(SEQ ID NO: 1)

tggt ccctgtcatt cactcgggtg cgtgtgtttg
tatgtctgtgtatgtatagg ggaaagaagg gatccctaac
tgttccctta tctgttttct acctcctcctttgtttaata
aaggctgaag ctttttgtac tcatgaaaaa aaaaaaaaaa aaa
shRNA sequence 1 targeting endogenous AURKB 3′UTR:
Top strand:

(SEQ ID NO: 2)

CACCG*TGTCATTCACTCGGGTGCGTGTGTTCGAAAACACACGCACCCGA
GTGAATGACA
Buttom strand:

(SEQ ID NO: 3)

AAAATGTCATTCACTCGGGTGCGTGTGTTTTCGAACACACGCACCCGAGT
GAATGACAC*
shRNA sequence 2: targeting endogenous AURKB
3′UTR:
Top strand:

(SEQ ID NO: 4)

CACCG*TCATTCACTCGGGTGCGTGTGTTTGCGAACAAACACACGCACCC
GAGTGAATGA
Buttom strand:

(SEQ ID NO: 5)

AAAATCATTCACTCGGGTGCGTGTGTTTGTTCGCAAACACACGCACCCGA
GTGAATGAC
shRNA sequence 3 targeting endogenous AURKB 3′UTR:
Top strand:

(SEQ ID NO: 6)

CACCGAAGGGATCCCTAACTGTTCCCTTACGAATAAGGGAACAGTTAGGG
ATCCCTTC-
Bottom strand:

(SEQ ID NO: 7)

AAAAGAAGGGATCCCTAACTGTTCCCTTATTCGTAAGGGAACAGTTAGGG
ATCCCTTC-
AURKB supplementary expression cassette 3′UTR
(SV40 PolyA):

(SEQ ID NO: 8)

Aacttgtttattgcagcttataatggttacaaataaagcaatagcatcac
aaatttcacaaataaagcatttttttcactgcattctagttgtggtttgt
ccaaactcatcaatgtatcttatcatgtctg

### Example 2

Essential gene knock-down cassette and supplementary expression cassette for AURKB gene. The gene knock-down cassette was targeted at ORF of endogenous AURKB, while supplementary AURKB expression cassette use codon optimized ORF to get rid of the shRNA target sequences

Endogenous AURKB shRNA targeeted sequences 1:

(SEQ ID NO: 9)

AatAGCAGTGGGACACCCGACATCTTAAcgcg
shRNA sequence 1 targeting endogenous AURKB ORF:
Top strand:

(SEQ ID NO: 10)

CACCAGCAGTGGGACACCCGACATCTTAACGAATTAAGATGTCGGGTGTC
CCACTGC-
Bottom strand:

(SEQ ID NO: 11)

AAAAGCAGTGGGACACCCGACATCTTAATTCGTTAAGATGTCGGGTGTCC
CACTGCT-
AURKB supplementary expression cassette modified
ORF:

(SEQ ID NO: 12)

AacTCCTCCGGAACGCCAGATATTCTCAcacg
Endogenous AURKB shRNA targeeted sequences 2:

(SEQ ID NO: 13)

TcccAACATCCTGCGTCTCTACAACTATTttta
shRNA sequence 2 targeting endogenous AURKB ORF:
Top strand:

(SEQ ID NO: 14)

CACCAACATCCTGCGTCTCTACAACTATTCGAAAATAGTTGTAGAGACGC
AGGATG
Bottom strand:

(SEQ ID NO: 15)

AAAACATCCTGCGTCTCTACAACTATTTTCGAATAGTTGTAGAGACGCAG
GATGTT
AURKB supplementary expression cassette modified
ORF:

(SEQ ID NO: 16)

TcctAAtATTCTAAGATTATACAATTACTtcta
shRNA targeeted Endogenous AURKB sequences:

(SEQ ID NO: 17)

GatcGCCGCCGCATTTATCTCATACTGGAatat
shRNA sequence 3 targeting endogenous AURKB ORF:
Top strand:

(SEQ ID NO: 18)

CACCGGAGGAGGATCTACTTGATTCTAGACGAATCTAGAATCAAGTAGAT
CCTCCTCC
Bottom strand:

(SEQ ID NO: 19)

AAAAGGAGGAGGATCTACTTGATTCTAGATTCGTCTAGAATCAAGTAGAT
CCTCCTCC
AURKB supplementary expression cassette modified
ORF:

(SEQ ID NO: 20)

GaccGGAGGAGGATCTACTTGATTCTAGAgtat

### Example 3

Essential gene knock-down cassette and supplementary expression cassette for Yes1 gene. The gene knock-down cassette was targeted at 3′UTR of endogenous Yes1, while supplementary expression cassette use BGH PolyA

Endogenous Yes1 shRNA targeted sequences 1:

(SEQ ID NO: 21)

CAGCCTACATTCACTTCTAAAGTCT
shRNA sequence 1 targeting endogenous Yes1 3′UTR:
Top strand:

(SEQ ID NO: 22)

CACCG*CAGCCTACATTCACTTCTAAAGTCTCGAAAGACTTTAGAAGTGA
ATGTAGGCTG
Bottom strand:

(SEQ ID NO: 23)

AAAACAGCCTACATTCACTTCTAAAGTCTTTCGAGACTTTAGAAGTGAAT
GTAGGCTGC
Endogenous Yes1 shRNA targeted sequences 2:

(SEQ ID NO: 24)

AGCCTACATTCACTTCTAAAGTCTA
shRNA sequence 1 targeting endogenous Yes1 3′UTR:
Top strand:

(SEQ ID NO: 25)

CACCAGCCTACATTCACTTCTAAAGTCTACGAATAGACTTTAGAAGTGAA
TGTAGGC
Bottom strand:

(SEQ ID NO: 26)

AAAAGCCTACATTCACTTCTAAAGTCTATTCGTAGACTTTAGAAGTGAAT
GTAGGCT
Endogenous Yes1 shRNA targeted sequences 3:

(SEQ ID NO: 27)

GCAGCATTAGTGGTTTGAGTGTCTA
shRNA sequence 1 targeting endogenous Yes1 3′UTR:
Top strand:

(SEQ ID NO: 28)

CACCGCAGCATTAGTGGTTTGAGTGTCTACGAATAGACACTCAAACCACT
AATGCTGC
Bottom strand:

(SEQ ID NO: 29)

AAAAGCAGCATTAGTGGTTTGAGTGTCTATTCGTAGACACTCAAACCACT
AATGCTGC
Yes1 supplementary expression cassette BGH PolyA:

(SEQ ID NO: 30)

Ctgtgccttctagttgccagccatctgttgtttgcccctcccccgtgcc
ttccttgaccctggaaggtgccactcccactgtcctttcctaataaaat
gaggaaattgcatcgcattgtctgagtaggtgtcattctattctggggg
gtggggtggggcaggacagcaagggggaggattgggaagacaatagcag
gcatgctggggatgcggtgggctctatgg

### Example 4

Essential gene knock-down cassette and supplementary expression cassette for Yes1 gene. The gene knock-down cassette was targeted at ORF of endogenous Yes1, while supplementary Yes1 expression cassette use codon optimized ORF to get rid of the shRNA target sequences

Endogenous yes1 shRNA targeted sequences 1:

(SEQ ID NO: 31)

CAGTTAATTTCAGCAGTCTTTCCAT
shRNA sequence 1 targeting endogenous Yes1 ORF:
Top strand:

(SEQ ID NO: 32)

CACCG*CAGTTAATTTCAGCAGTCTTTCCATCGAAATGGAAAGACTGCTG
AAATTAACTG
Bottom strand:

(SEQ ID NO: 33)

AAAACAGTTAATTTCAGCAGTCTTTCCATTTCGATGGAAAGACTGCTGAA
ATTAACTGC
Yes1 supplementary expression cassette modified
ORE:

(SEQ ID NO: 34)

CTGTAAACTTTTCCTCCTTAAGCAT
Endogenous Yes1 shRNA targeted sequences 2:
shRNA sequence 2 targeting endogenous Yes ORF:

(SEQ ID NO: 35)

GCCAAGTTCATATCCTGCTGGTTTA
Top strand:

(SEQ ID NO: 36)

CACCGCCAAGTTCATATCCTGCTGGTTTACGAATAAACCAGCAGGATATG
AACTTGGC
Bottom strand:

(SEQ ID NO: 37)

AAAAGCCAAGTTCATATCCTGCTGGTTTATTCGTAAACCAGCAGGATATG
AACTTGGC
Yes1 supplementary expression cassette modified
ORF:

(SEQ ID NO: 38)

ACCTAGCAGCTACCCAGCAGGACTT
Endogenous Yes1 shRNA targeted sequences 3:
shRNA sequence 3 targeting endogenous yes1 ORF:

(SEQ ID NO: 39)

GCTGATGGTTTATGCCACAAGTTGA
Top strand:

(SEQ ID NO: 40)

CACCGCTGATGGTTTATGCCACAAGTTGACGAATCAACTTGTGGCATAAA
CCATCAGC
Bottom strand:

(SEQ ID NO: 41)

AAAAGCTGATGGTTTATGCCACAAGTTGATTCGTCAACTTGTGGCATAAA
CCATCAGC
Yes1 supplementary expression cassette modified
ORF:

(SEQ ID NO: 42)

GCAGACGGACTGTGTCATAAACTTA

### Example 5

Essential gene knock-down cassette and supplementary expression cassette for WDR12 gene. The gene knock-down cassette was targeted at 3′UTR of endogenous WDR12, while supplementary expression cassette use BGH PolyA

Endogenous WDR12 shRNA targeted sequences 1:

(SEQ ID NO: 43)

AATGAAATTGGTAGAGAACCATGAA
shRNA sequence 1 targeting endogenous WDR12
3′UTR:
Top strand:

(SEQ ID NO: 44)

CACCAATGAAATTGGTAGAGAACCATGAACGAATTCATGGTTCTCTACCA
ATTTCA
Bottom strand:

(SEQ ID NO: 45)

AAAATGAAATTGGTAGAGAACCATGAATTCGTTCATGGTTCTCTACCAAT
TTCATT
Endogenous WDR12 shRNA targeted sequences 2:
shRNA sequence 1 targeting endogenous WDR12 3′UTR:

(SEQ ID NO: 46)

TGAAATTGGTAGAGAACCATGAAAT
Top strand:

(SEQ ID NO: 47)

CACCG*TGAAATTGGTAGAGAACCATGAAATCGAAATTTCATGGTTCTCT
ACCAATTTCA
Bottom strand:

(SEQ ID NO: 48)

AAAATGAAATTGGTAGAGAACCATGAAATTTCGATTTCATGGTTCTCTAC
CAATTTCAC
Endogenous WDR12 shRNA targeted sequences 3:
shRNA sequence 1 targeting endogenous DR12 UTR:

(SEQ ID NO: 49)

GAAATTGGTAGAGAACCATGAAATT
Top strand:

(SEQ ID NO: 50)

CACCGAAATTGGTAGAGAACCATGAAATTCGAAAATTTCATGGTTCTCTA
CCAATTTC
Bottom strand:

(SEQ ID NO: 51)

AAAAGAAATTGGTAGAGAACCATGAAATTTTCGAATTTCATGGTTCTCTA
CCAATTTC
WDR12 supplementary expression cassette BGH PolyA:

(SEQ ID NO: 52)

Ctgtgccttctagttgccagccatctgttgtttgcccctcccccgtgcct
tccttgaccctggaaggtgccactcccactgtcctttcctaataaaatga
ggaaattgcatcgcattgtctgagtaggtgtcattctattctggggggtg
gggtggggcaggacagcaagggggaggattgggaagacaatagcaggcat
gctggggatgcggtgggctctatgg

I claim:

1. A method to generate cell clones with stable transgene (Gene Of Interest) expression. The method are composed of
(a.) Inactivation of endogenous essential genes of cell substrate through Gene Inactivation Cassettes (GIC)
(b.) Trans-complementation of essential genes products through Gene Supplementary Cassettes (GSC), alternatively, the cassette codes for Gene product that capable of inactivating GIC function
(c.) The GOI expression cassettes are preferably, not necessary, physically linked to GSC to take advantage of favorable selective pressure.
(d.) Gene Inactivation Cassettes could be preferably, not necessary, physically linked to Gene Supplementary Cassettes and GOI expression cassettes

2. The composition of claim 1 (a), the endogenous essential genes of cell substrate refer to gene or combinations of genes that are essential for cell survive and/or cell proliferation.

3. The composition of claim 1 (a), the first recombinant DNA molecules (GIC) comprising a vector, said vector comprising of a promoter operably linked to RNAi (shRNA)/dsRNA/ribozyme molecule, or tandem repeats of such transcripts units, targeted to cell endogenous essential mRNA.

4. The composition of claim 3, where said promoter comprise
A. PolIII promoters include but not limited to, U6, H1, 7SK, EBV-encoded small RNAs (EBERs) and adenovirus VAI promoters, etc.
B. PolII promoter, include but not limited to, CMV promoter, RSV promoter, retroviral LTR elements, etc.
C. Poll promoter

5. The composition of claim 3, where in said shRNA/siRNA/dsRNA/ribozyme comprising nucleotide sequences sufficiently complementary to targeted cell endogenous essential mRNAs to mediate degradation of said targets.

6. The composition of claim 2, where in said targeted cell endogenous essential gene mRNA encodes various strains of viral transforming proteins or their combinations, include but not limited to, adenovirus E1a/1b, SV40 large T antigen, Papillomaviruses E6 and E7, Epstein-Barr (EBNA1) virus, etc.

7. The composition of claim 2, where in said target mRNAs encode endogenous cell essential gene or genes of various cell types from various organisms, include but not limited to, genes crucial for cell cycles/metabolism, include but not limit to FGFs, CDC2, MYC, E2F, CDC45L, PCNA, RRM1, RRM2, CHAF1A, SLBP and NPAT, etc.

8. The composition of claim 2, where in said target mRNAs encode endogenous cell essential gene or genes, include, but not limited to, sequences selected from SEQ ID NO: 1-2509 and their homologous sequences from various organisms.

9. The composition of claim 1 (b), a second recombinant DNA vector, said vector comprising of a promoter operable linked to the supplementary mRNA coding for functional essential gene products similar to inactivated endogenous counter part and elements necessary for transcript stability and translation enhancement.

10. The composition of claim 1 (b), the GSC is physically linked to, or separates from, GOI expression cassette. The supplementary essential genes and gene of interests (recombinant bioactive proteins) are expressed from two separate transcript units.

11. The composition of claim 1 (b), the GSC is physically linked to, or separates from, GOI expression cassette. The supplementary essential genes and gene of interests (recombinant bioactive proteins) are expressed from a polycistronic transcript, such as through IRES, intron vector, split gene strategy, or expressed from a bi-directional promoter.

12. The composition of claim 1(b), where said supplementary mRNA contains different 5′ or 3′ UTR sequences from that of endogenous counter part.

13. The composition of claim 1(b), where said supplementary mRNA coding region has been optimized through mutation, deletion or insertion to abolish the target sequences for GIC in claim 2.

14. The composition of claim 1(b), the said vector (GSC) is physically linked to, or separates from, GIC vector. The GSC and GIC may be transcribed from a bi-directional promoter, or GIC and GSC may be derived from a single transcription unit, or GIC may be embedded in the intron of GSC.

15. The GIC might target essential genel and GSC supplement with gene 2. The GSC is, preferably but not absolutely necessary, codes for a different (gene 2) as that of inactivated by GIC (gene 1) on the same DNA molecules (plasmid, etc.).

16. The composition of claim 1(b), the said vector (GSC) transcripts and selective marker protein are expressed from a polycistronic transcript, such as through IRES, intron vector, split gene strategy, or expressed from a bi-directional promoter.

17. The composition of claim 1(c), the said vector (GSC) said comprising of a promoter operably linked to RNAi (shRNA)/dsRNA/ribozyme molecule, or tandem repeats of such transcripts units, targeted to GIC transcripts.

18. The composition of claim 1(d), The GOI includes, but not limited to, antibodies, cytokines, hormones, recombinant coagulation factors, enzymes viral proteins and other bioactive recombinant proteins, etc.

19. The composition of claim 1, the Gene inactivation cassettes are physically linked to, or separate from, supplementary cassettes and GOI expression cassette.

20. The composition of claim 1, the Gene inactivation cassettes can target the homologous region of gene family or families.

21. The composition of claim 1, one or multiple sets of the said vectors (Gene inactivation cassette, supplementary cassette and GOI expression cassette) are introduced into same single cell substrate
